(19) United States
(12) Patent Application Publication
(10) Pub. No.: US 2024/0293572 A1
(43) Pub. Date: Sep. 5, 2024

(54) THERAPEUTIC-AGENT-RELEASING HYDROGELS AND PRECURSORS THEREOF

(71) Applicant: Boston Scientific Scimed, Inc., Maple Grove, MN (US)

(72) Inventors: Yen-Hao Hsu, Shrewsbury, MA (US); Cristian Parisi, Boston, MA (US); Joseph Thomas Delaney, JR., Minneapolis, MN (US)

(73) Assignee: Boston Scientific Scimed, Inc., Maple Grove, MN (US)

(21) Appl. No.: 18/590,582

(22) Filed: Feb. 28, 2024

Related U.S. Application Data
(60) Provisional application No. 63/487,959, filed on Mar. 2, 2023.

(51) Int. Cl. A61K 47/69; A61K 47/61; A61K 47/64

(52) U.S. Cl. CPC A61K 47/6903; A61K 47/61; A61K 47/64

(57) ABSTRACT

In some aspects, the present disclosure provides therapeutic-agent-releasing polyamino compounds that comprise a polyamino moiety that is covalently linked to a residue of a hydroxyl-substituted therapeutic agent by an ester bond. In various embodiments, such therapeutic-agent-releasing polyamino compounds are used to form therapeutic-agent-releasing hydrogels. In other aspects, the present disclosure provides therapeutic-agent-releasing polysaccharides that comprise a plurality of hydroxyl-substituted therapeutic agent residues that are covalently linked to a carboxylic-acid-containing polysaccharide along a backbone of the carboxylic-acid-containing polysaccharide.

### CROSS-REFERENCE TO RELATED APPLICATIONS

This application claims the benefit of U.S. Provisional Patent Application Ser. No. 63/487,959 filed on Mar. 2, 2023, the disclosure of which is incorporated herein by reference.

### FIELD

The present disclosure relates to therapeutic-agent-releasing compounds and hydrogels and to methods of making and using therapeutic-agent-releasing compounds and hydrogels, among other aspects.

### BACKGROUND

In vivo crosslinkable hydrogels, commercially available under the name of SpaceOAR®, are able to create space between prostate and rectum to reduce side effects during radiotherapies for prostate cancer treatment. This crosslinked hydrogel is based on multi-arm polymers functionalized with the activated ester as end groups, specifically, 8-arm PEG with succinimidyl glutarate end groups (PEG-SG), that further reacts with trilysine to quickly form the crosslinked networks. The crosslinked gel persists in the body for a minimum of three months during cancer therapy, then gradually degrades via hydrolysis in vivo.

Non-animal stabilized hyaluronic acid (NASHA) solutions form physically crosslinked hydrogels, suitable for injections and as fillers, with good biocompatibility. One hyaluronic acid-based hydrogel, available as Barrigel®, is used to minimize radiation-associated side effects during prostate cancer treatment by creating the space in between the prostate and rectum. Barrigel® stays in place for about 6 to 9 months and is then broken down by the body over time.

Neither of these products, however, is capable of therapeutic agent release.

There is a continuing need in the biomedical arts for therapeutic-agent-containing hydrogels, for methods of making and using such hydrogels, and for systems for forming such hydrogels, among other needs.

### SUMMARY

In various aspects, the present disclosure provides therapeutic-agent-releasing polyamino compounds that comprise a polyamino moiety that is covalently linked to a residue of a hydroxyl-substituted therapeutic agent by an ester bond. In various embodiments, hydrolysis of the ester bond results in formation of the hydroxyl-substituted therapeutic agent and a carboxyl-substituted polyamino compound.

In some embodiments, which are applicable to the above aspects and embodiments, the hydroxyl-substituted therapeutic agent comprises one or more primary hydroxyalkyl groups selected from hydroxymethyl, 2-hydroxyethyl, 3-hydroxypropyl, 4-hydroxybutyl, 5-hydroxypentyl, 6-hydroxyhexyl, 7-hydroxyheptyl and/or 8-hydroxyoctyl groups.

In some embodiments, which are applicable to the above aspects and embodiments, the polyamino moiety comprises a plurality of —(CH₂)ₓ—NH₂ groups where x is 0, 1, 2 3, 4, 5, 6, 7 or 8.

In some embodiments, which are applicable to the above aspects and embodiments, the polyamino moiety comprises a residue of a carboxyl-substituted polyamino compound.

In some embodiments, which are applicable to the above aspects and embodiments, the polyamino moiety comprises two or more amino acid residues selected from residues of lysine, ornithine, and combinations thereof.

In various aspects, the present disclosure provides systems for forming a hydrogel that comprise (a) a therapeutic-agent-releasing polyamino compound in accordance with any of the above aspects and embodiments and (b) a reactive multi-arm polymer that comprises a plurality of hydrophilic polymer arms having reactive end groups that are reactive with amino groups of the therapeutic-agent-releasing polyamino compound.

In some embodiments, the hydrophilic polymer arms comprise one or more hydrophilic monomers selected from ethylene oxide, N-vinyl pyrrolidone, oxazolines, hydroxyethyl acrylate, hydroxyethyl methacrylate, PEG methyl ether acrylate or PEG methyl ether methacrylate, or PNIPAAM.

In some embodiments, which are applicable to the above aspects and embodiments, the reactive end groups are linked to the hydrophilic polymer arms by a hydrolysable ester and/or wherein the reactive end groups are electrophilic groups.

In some embodiments, which are applicable to the above aspects and embodiments, the electrophilic groups are selected from imidazole esters, imidazole carboxylates, benzotriazole esters, or imide esters.

In some embodiments, which are applicable to the above aspects and embodiments, the system comprises a first composition that comprises the therapeutic-agent-releasing polyamino compound, a second composition that comprises the reactive multi-arm polymer, and an optional accelerant composition.

In some embodiments, which are applicable to the above aspects and embodiments, the systems further comprise a delivery device.

In various aspects, the present disclosure provides medical hydrogels that are formed by crosslinking a therapeutic-agent-releasing polyamino compound in accordance with any of the above aspects and embodiments and a reactive multi-arm polymer that comprises a plurality of hydrophilic polymer arms having reactive end groups that are reactive with amino groups of the therapeutic-agent-releasing polyamino compound.

In various aspects, the present disclosure provides methods of treatment comprising administering to a subject a mixture that comprises a therapeutic-agent-releasing polyamino compound in accordance with the above aspects and embodiments and a reactive multi-arm polymer that comprises a plurality of hydrophilic polymer arms having reactive end groups that are reactive with amino groups of the therapeutic-agent-releasing polyamino compound under conditions such that the therapeutic-agent-releasing polyamino compound and the reactive multi-arm polymer cross-link after administration to the subject.

In various aspects, the present disclosure provides methods of making therapeutic-agent-releasing polyamino compounds that comprise (a) forming a protected carboxyl-substituted polyamino compound by protecting amino groups of the carboxyl-substituted polyamino compound, (b) forming an ester linkage between the carboxyl group of the protected carboxyl-substituted polyamino compound and a primary hydroxyl group of a hydroxyl-containing therapeutic agent and (c) deprotecting amino groups of the product of step (b).

In various aspects, the present disclosure provides therapeutic-agent-releasing polysaccharides that comprise a plurality of hydroxyl-substituted therapeutic agent residues that are covalently linked to a carboxylic-acid-containing polysaccharide along a backbone of the carboxylic-acid-containing polysaccharide. In some embodiments the hydroxyl-substituted therapeutic agent residues are covalently linked to the carboxylic-acid-containing polysaccharide through ester bonds.

In various aspects, the present disclosure provides therapeutic-agent-releasing polysaccharides that comprise a carboxylic-acid-containing polysaccharide that is crosslinked through amide bonds by a therapeutic-agent-releasing polyamino compound in accordance with any of the above aspects and embodiments.

In some embodiments, which are applicable to the above aspects and embodiments, the carboxylic-acid-containing polysaccharide comprises one or more uronic acid species selected from galacturonic acid, glucuronic acid, and iduronic acid.

In some embodiments, which are applicable to the above aspects and embodiments, the carboxylic-acid-containing polysaccharide is selected from hyaluronic acid, alginic acid, pectin, agaropectin, carrageenan, gellan gum, gum arabic, guar gum, xanthan gum, and carboxymethyl cellulose.

In various aspects, the present disclosure provides kits that comprise a therapeutic-agent-releasing polysaccharide in accordance with any of the above aspects and embodiments in a reservoir. In some embodiments, the reservoir is a syringe barrel.

In various aspects, the present disclosure provides methods of treatment comprising administering to a subject a therapeutic-agent-releasing polysaccharide in accordance with any of the above aspects and embodiments.

In various aspects, the present disclosure provides methods of making a therapeutic-agent-releasing polysaccharide compound comprising reacting a primary hydroxyl group of a hydroxyl-substituted therapeutic agent with carboxylic acid groups of a carboxylic-acid-containing polysaccharide in an ester coupling reaction.

One advantage of the present disclosure is that hydrogels are provided that act as prodrugs in that, after being established in vivo, the hydrogels undergo chemical conversion by metabolic processes (including hydrolysis) with one of the products of the chemical conversion being a therapeutic agent (also referred to herein as a “drug”) in pharmacologically active form.

The above and other aspects, embodiments, features and benefits of the present disclosure will be readily apparent from the following detailed description.

### BRIEF DESCRIPTION OF THE DRAWINGS

FIG. 1 schematically illustrates a method of forming a therapeutic-agent-releasing polyamino compound, in accordance with an embodiment of the present disclosure.

FIG. 2 schematically illustrates ester bond hydrolysis of the therapeutic-agent-releasing polyamino compound of FIG. 1.

FIG. 3 schematically illustrates a method of forming a therapeutic-agent-releasing hydrogel, in accordance with an embodiment of the present disclosure.

FIG. 4 schematically illustrates a method of forming a therapeutic-agent-releasing hydrogel, in accordance with another embodiment of the present disclosure.

### DETAILED DESCRIPTION

In some aspects of the present disclosure, therapeutic-agent-releasing polyamino compounds are provided that comprise a polyamino moiety that is covalently linked to a therapeutic agent precursor by an ester bond. Upon hydrolysis of the ester bond, therapeutic-agent-releasing polyamino compounds release the therapeutic agent.

In various embodiments, the therapeutic agent precursor is a precursor of a hydroxyl-substituted therapeutic agent. In these embodiments, upon hydrolysis of the ester bond, the therapeutic-agent-releasing polyamino compound releases the hydroxyl-substituted therapeutic agent. As seen below, in various embodiments, the therapeutic agent precursor is a residue of a hydroxyl-substituted therapeutic agent, which may be formed, for example, by an ester-forming reaction between a hydroxyl-substituted therapeutic agent and a carboxyl-substituted polyamino compound.

As defined herein, hydroxyl-substituted therapeutic agents are therapeutic agents that have one or more hydroxyl groups, typically, therapeutic agents having one or more primary alcohol groups (i.e., one or more —CH₂OH groups). In some embodiments, the therapeutic agents have one or more primary hydroxyalkyl groups, for example, one or more primary C₁-C₈-hydroxyalkyl groups, including therapeutic agents having one or more —(CH₂)ₓOH groups where x is 1, 2, 3, 4, 5, 6, 7 or 8 (e.g., therapeutic agents having one or more hydroxymethyl, 2-hydroxyethyl, 3-hydroxypropyl, 4-hydroxybutyl, 5-hydroxypentyl, 6-hydroxyhexyl, 7-hydroxyheptyl and/or 8-hydroxyoctyl groups.

Examples of hydroxyl-substituted therapeutic agents include hydroxyl-substituted antimetabolites, hydroxyl-substituted alkylating agents, hydroxyl-substituted antibiotics, hydroxyl-substituted microtubule inhibitors, hydroxyl-substituted hormones, hydroxyl-substituted hormone antagonists, hydroxyl-substituted monoclonal antibodies, hydroxyl-substituted antimitotics, hydroxyl-substituted immunosuppressive agents, hydroxyl-substituted tyrosine and serine/threonine kinases, hydroxyl-substituted proteasome inhibitors, hydroxyl-substituted matrix metalloproteinase inhibitors, hydroxyl-substituted Bcl-2 inhibitors, hydroxyl-substituted DNA alkylating agents, hydroxyl-substituted spindle poisons, hydroxyl-substituted poly (DP-ribose)polymerase (PARP) inhibitors, and combinations thereof.

Particular examples of hydroxyl-substituted therapeutic agents include cladribine, cytarabine, floxuridine, fludarabine gemcitabine, bleomycin, doxorubicin, epirubicin, streptozocin, leuprolide, and prednisone, among many others.

In various embodiments, the therapeutic-agent-releasing polyamino compounds of the present disclosure comprise a polyamino moiety having a plurality (two, three, four, five, six, seven, eight, nine, ten or more) primary amine groups. For example, the polyamino moiety may comprises a plurality of (two, three, four, five, six, seven, eight, nine, ten or more) —(CH₂)ₓ—NH₂ groups where x is 0, 1, 2, 3, 4, 5 or 6. In some of these embodiments, the polyamino moiety may comprises a plurality of —(CH₂)ₓ—NH₂ groups disposed along a polymeric moiety (defined herein as a moiety comprising 2, 3, 4, 5, 6, 7, 8, 9, 10, 20, 50, 100, 200, 500, 1000 or more or more monomer residues/units). In some embodiments, the polymeric moiety may be selected from a polyamide moiety, such as a peptide moiety, a polyalkylene moiety, or a polysaccharide moiety, among others.

In some embodiments, the polyamino moiety of the therapeutic-agent-releasing polyamino compounds may correspond to a residue of a carboxyl-substituted polyamino compound (i.e., a compound comprising a carboxyl group and a plurality of amino groups). Depending on the pH, the carboxyl group may be in the form of a carboxylic acid group or an anionic carboxylate group.

Examples of carboxyl-substituted polyamino compounds include peptides that contain two or more amino acid residues that have one or more primary amine groups, for example, two or more amino acid residues that each comprise one or more —(CH₂)ₓ—NH₂ groups, where x is 0, 1, 2, 3, 4, 5, 6, 7 or 8. As used herein, an “amino acid” is an organic compound that contain an amino group (—NH₂), a carboxylic acid group (—COOH), and a side group that is specific to each amino acid. Depending on the surrounding pH, the amino group may be positively charged (—NH₃⁺) and/or the carboxylic acid group may be negatively charged (—COO). Examples of amino acids include alpha-amino acids, beta-amino acids, gamma-amino acids, delta-amino acids, epsilon-amino acids, and so forth.

Examples of carboxyl-substituted polyamino compounds include peptides comprising from 2 to 10 lysine and/or ornithine amino acid residues, including polylysine peptides (e.g., dilysine, trilysine, tetralysine, pentalysine, etc.), polyornithine peptides (e.g., diornithine, triornithine, tetraornithine, pentaornithine, etc.), and poly(lysine-co-ornithine) peptides. Examples of carboxyl-substituted polyamino compounds further include carboxyl-terminated polyamines such as carboxyl-terminated poly(allyl amine), carboxyl-terminated poly(vinyl amine), and carboxyl-terminated chitosan.

Commercially available examples of carboxyl-substituted polyamino compounds also include 16-amino-3-[2-[(4-aminobutyl)(3-aminopropyl)amino]-2-oxoethyl]-12-(3-aminopropyl)-6,9-bis(carboxymethyl)-11-oxo-3,6,9,12-tetraazahexadecanoic acid, L-ornithyl-L-ornithyl-L-ornithine, N2-[1-[N2-[N2-(N-L-valyl-L-alanyl)-L-lysyl]-L-lysyl]-L-prolyl]-L-Lysine, L-Lysyl-L-tryptophyl-L-lysyl-L-lysine, N²,N⁵,N⁵-tris(3-aminopropyl)-L-ornithine, L-lysyl-L-ornithyl-L-lysine, D-lysyl-D-lysyl-D-lysine, glycylglycyl-L-lysylglycylglycyl-L-lysine, N²-[N⁴-[N-[N-(N-glycylglycyl)glycyl]glycyl]-L-lysyl]-L-lysine, L-Lysyl-L-threonyl-L-lysyl-L-lysine, glycylglycyl-L-lysyl-L-lysylglycyl-L-cysteine, L-lysyl-L-arginyl-L-lysyl-L-lysine, L-arginyl-L-lysyl-L-lysyl-L-lysine, L-leucyl-L-lysyl-L-seryl-L-lysyl-L-lysine, L-alanyl-L-methionylglycyl-L-lysyl-L-lysyl-L-lysine, L-lysyl-L-lysyl-L-lysyl-L-arginyl-L-glutamine, L-seryl-L-isoleucyl-L-lysyl-L-lysyl-Llysyl-L-lysine, N²-(N²-L-ornithyl-L-lysyl)-L-lysine, L-lysyl-L-lysyl-L-lysine, and L-lysyl-L-lysyl-L-lysyl-L-alanine, among many others.

As previously indicated, in various embodiments, the therapeutic-agent-releasing polyamino compounds of the present disclosure comprise a residue of a carboxyl-substituted polyamino compound that is covalently linked to a residue of a hydroxyl-substituted therapeutic agent by an ester bond. Upon hydrolysis of the ester bond, the hydroxyl-substituted therapeutic agent and the carboxyl-substituted polyamino compound are released from one another and reestablished as independent chemical entities.

The therapeutic-agent-releasing polyamino compounds of the present disclosure may be formed by an ester coupling reaction between a carboxyl group of a carboxyl-substituted polyamino compound, which may be selected, for example, from those described above (after protection of the amino groups), and a hydroxyl group of a hydroxyl-containing therapeutic agent, which may be selected, for example, from those described above. The resulting therapeutic-agent-releasing polyamino compounds therefore comprise a residue of the carboxyl-substituted polyamino compound that is linked to a residue of the hydroxyl-substituted therapeutic agent by an ester bond. Upon hydrolysis of the ester bond, the hydroxyl-substituted therapeutic agent is released and the carboxyl group of the residue of the carboxyl-substituted polyamino compound is regenerated.

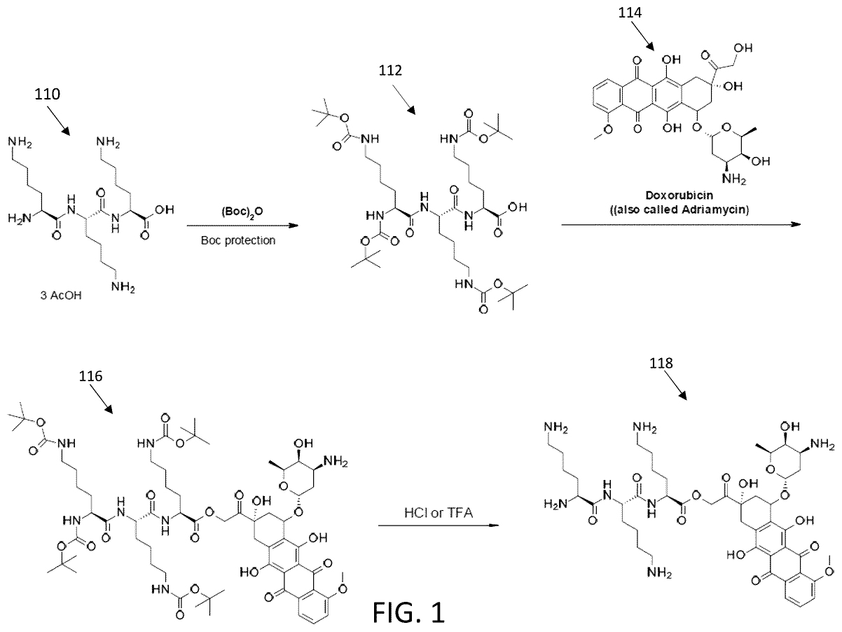

More particularly, in a first step, amino groups of a carboxyl-substituted polyamino compound may be protected with a suitable protective agent. The amino groups are protected for compatibility with other reactants in a subsequent ester coupling reaction. For example, amino groups of a carboxyl-substituted polyamino compound may be protected by reaction with di-tert-butyl dicarbonate, (Boc)₂O. In a particular example, and with reference to FIG. 1, amino groups of trilysine (110) are protected using di-tert-butyl dicarbonate (CAS #24424-99-5), thereby forming Boc-protected trilysine (112). This leaves the carboxyl group of the protected compound (Boc-protected trilysine) available for ester coupling.

In a second step, a hydroxyl-containing therapeutic agent is coupled with the protected carboxyl-substituted polyamino compound as formed in the first step in an ester coupling reaction (e.g., via a carbodiimide coupling reagent) to form a Boc-protected therapeutic-agent-releasing polyamino compound. For example, with continued reference to FIG. 1, Boc-protected trilysine (112) may be reacted in an ester coupling reaction with a doxorubicin (114) (a hydroxy-methyl substituted therapeutic agent) to form a Boc-protected trilysine-doxorubicin conjugate (116).

In a third step, deprotection of the protected carboxyl-substituted polyamino compound product of the second step is performed (e.g., under acidic conditions) to form a final therapeutic-agent-releasing polyamino compound. For example, with further reference to FIG. 1, Boc-protected trilysine-doxorubicin conjugate (Boc-TL-Dox) (116) formed in the second step is deprotected under acidic conditions (e.g., using an acid such as hydrochloric acid or trifluoroacetic acid) to form a therapeutic-agent-releasing polyamino compound, specifically, a trilysine-doxorubicin conjugate (TL-Dox) (118).

The process described above can be performed using a variety of carboxyl-substituted polyamino compounds and a variety of hydroxyl-containing therapeutic agents.

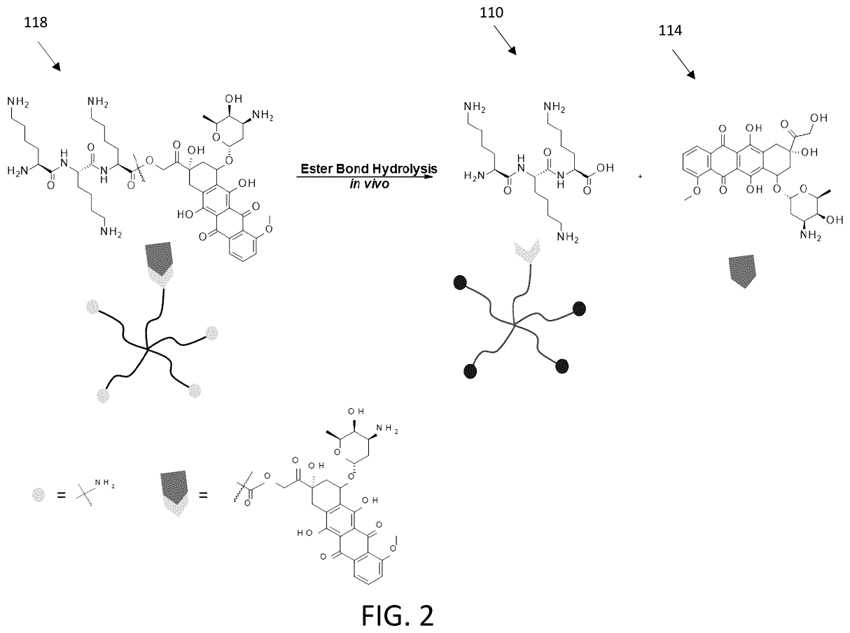

In some embodiments, the therapeutic-agent-releasing polyamino compound may be independently used as a source of therapeutic agent. For example, as shown in FIG. 2, upon hydrolysis of the ester bond in the trilysine-doxorubicin conjugate (118) of FIG. 1, doxorubicin (114) and trilysine (110) are released from one another and reestablished as independent chemical entities.

However, as detailed below, in certain preferred embodiments, the therapeutic-agent-releasing polyamino compound is used to form a therapeutic-agent-releasing hydrogel.

For example, in some aspects of the present disclosure, therapeutic-agent-releasing hydrogels are provided that comprise a crosslinked reaction product of (a) a therapeutic-agent-releasing polyamino compound as described above and (b) a reactive multi-arm polymer that comprises a plurality of reactive end groups that are reactive with the amino groups of the therapeutic-agent-releasing polyamino compound. Unless indicated otherwise, as used herein the prefix “poly” means 2 or more (e.g., including 2, 3, 4, 5, 6, 7, 8, 9, 10, 20, 50, 100, 200, 500, 1000 or more).

In various embodiments, the reactive end groups of the reactive multi-arm polymer and the amino groups of the therapeutic-agent-releasing polyamino compound react with one another form a crosslinked product. The reactive multi-arm polymer may be water soluble.

Reactive multi-arm polymers for use herein include those that comprise a plurality of polymer arms (e.g., having two, three, four, five, six, seven, eight, nine, ten, eleven, twelve or more arms), wherein two or more polymer arms of the multi-arm polymers comprise one or more reactive end groups. In some embodiments, compositions containing the reactive multi-arm polymers may be provided in which a percentage of the polymer arms comprising one or more reactive end groups may correspond to between 50% and 100% of the total number of polymer arms in the composition (e.g., ranging anywhere from 50% to 70% to 80% to 90% to 95% to 99% to 100% of the total number of polymer arms). Typical average molecular weights for the reactive multi-arm polymers for use herein are of at least 10 kDa, in some cases ranging from 10 kDa to 50 kDa or more. In various embodiments, the reactive multi-arm polymers for use herein have a melting point of 40° C. or greater, preferably 45° C. or greater.

In various embodiments, the polymer arms are hydrophilic polymer arms. Such hydrophilic polymer arms may be composed of any of a variety of synthetic, natural, or hybrid synthetic-natural polymers including, for example, poly(alkylene oxides) such as poly(ethylene oxide) (PEO, also referred to as polyethylene glycol or PEG), poly(propylene oxide) or poly(ethylene oxide-co-propylene oxide), poly(N-vinyl pyrrolidone), polyoxazolines including poly(2-alkyl-2-oxazolines) such as poly(2-methyl-2-oxazoline), poly(2-ethyl-2-oxazoline) and poly(2-propyl-2-oxazoline), poly(vinyl alcohol), poly(allyl alcohol), polyhydroxyethyl acrylate, polyhydroxyethyl methacrylate, PEG methyl ether acrylate or PEG methyl ether methacrylate, or PNIPAAM, polysaccharides, and combinations thereof.

In various embodiments, the polymer arms extend from a core region. In certain embodiments, the core region comprises a residue of a polyol that is used to form the polymer arms. Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, tripentaerythritol adonitol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl)alkanes, such as 1,1,1-tris(4-hydroxyphenyl)ethane, and 2,6-bis(hydroxyalkyl)cresols, among others. In certain beneficial embodiments, the core region comprises a residue of a polyol that contains two, three, four, five, six, seven, eight, nine, ten or more hydroxyl groups.

In certain embodiments, the core region comprises a silsesquioxane. A silsesquioxane is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T =the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

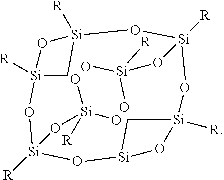

In the present disclosure, at least two R groups comprise polymer arms, and typically all R groups comprise polymer arms.

In certain embodiments, the reactive end groups may be electrophilic groups selected from imidazole esters, imidazole carboxylates, benzotriazole esters, or imide esters, including N-hydroxysuccinimidyl esters. A particularly beneficial reactive end group is an N-hydroxysuccinimidyl ester group. In certain embodiments, the reactive end groups are linked to the polymer arms via hydrolysable ester groups. Hydrolysable ester groups may be selected, for example, from glutarate ester groups, succinate ester groups, carbonate ester groups, or adipate ester groups. In particular embodiments, the polymer arms may be terminated with the following reactive, hydrolysable groups, among others: succinimidyl glutarate groups, succinimidyl succinate groups, succinimidyl carbonate groups, or succinimidyl adipate groups, among others.

Further examples of reactive multi-arm polymers are described, for example, in U.S. Patent Application Nos. 2011/0142936, 2021/0061950, 2021/0061954 and 2021/0061957.

In some aspects of the present disclosure, systems are provided that are configured to deliver (a) a therapeutic-agent-releasing polyamino compound and (b) a reactive multi-arm polymer that comprises a plurality of reactive end groups that are reactive with the amino groups of the therapeutic-agent-releasing polyamino compound under conditions such that the therapeutic-agent-releasing polyamino compound and the reactive multi-arm polymer crosslink with one another. In certain embodiments, those conditions comprise an environment having a basic pH, for example, a pH ranging from about 9 to about 11, typically ranging from about 9.5 to about 10.5, and more typically ranging from about 9.8 to about 10.2. Such systems can be used to form crosslinked hydrogels, either in vivo or ex vivo.

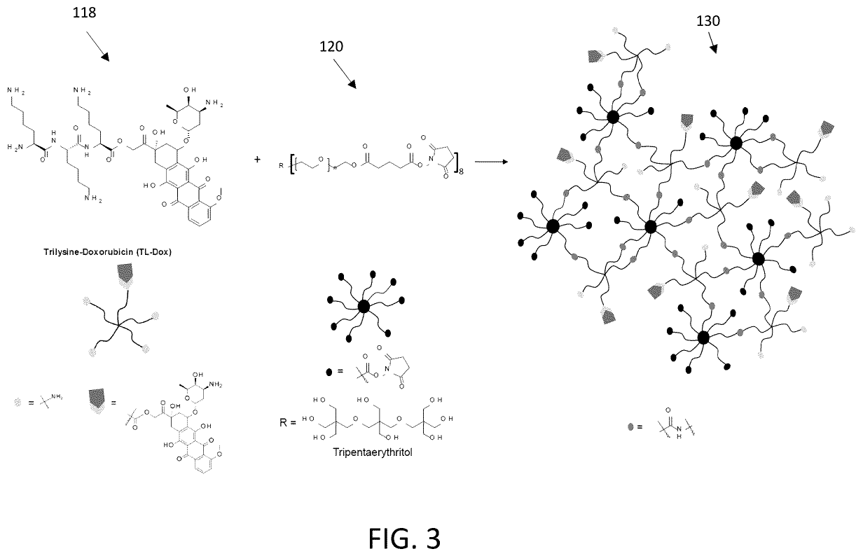

For example, as shown schematically in FIG. 3, a reactive multi-arm polymer like that described above, specifically a reactive multi-arm polymer (120) which comprises a core region R (for example, a tripentaerythritol residue core) and a plurality of hydrophilic polyethylene oxide arms (specifically, eight polyethylene oxide arms, where n ranges, for example, from 25 to 140) having reactive end groups (i.e., succinimidyl glutarate groups) can be crosslinked with a therapeutic-agent-releasing polyamino compound (118) like that described above, specifically a trilysine-doxorubicin conjugate (118), which comprises amino groups that are reactive with the reactive groups (i.e., succinimidyl glutarate groups) of the reactive multi-arm polymer by reacting the therapeutic-agent-releasing polyamino compound with the reactive multi-arm polymer under basic conditions, to form a crosslinked product (130), which may be in the form of a hydrogel when hydrated.

An advantage to this approach is that the therapeutic agent release is separate from the parent polymer, and the multi-arm polymer can be swapped out with N-hydroxysuccinimidyl-ester-functionalized systems having hydrophilic polymer arms other than polyethylene oxide arms, for example, synthetic, natural, or hybrid synthetic-natural hydrophilic polymer arms such as those described above.

In some aspects of the present disclosure, a system is provided that comprises (a) a first composition that comprises a therapeutic-agent-releasing polyamino compound and (b) a second composition that comprises a reactive multi-arm polymer that comprises a plurality of reactive end groups that are reactive with the amino groups of the therapeutic-agent-releasing polyamino compound.

The first composition may be a first fluid composition comprising the therapeutic-agent-releasing polyamino compound or a first dry composition that comprises the therapeutic-agent-releasing polyamino compound, to which a suitable fluid such as water for injection, saline, etc. can be added to form a first fluid composition. In addition to the therapeutic-agent-releasing polyamino compound, the first composition may further comprise additional agents, including those described below.

The second composition may be a second fluid composition comprising the reactive multi-arm polymer or a second dry composition that comprises the reactive multi-arm polymer, to which a suitable fluid such as water for injection, saline, etc. can be added to form a second fluid composition. In addition to the reactive multi-arm polymer, the second composition may further comprise additional agents including as those described below.

In some embodiments, the therapeutic-agent-releasing polyamino compound (118) is initially combined with the reactive multi-arm polymer (120) at an acidic pH at which crosslinking between the reactive groups of the reactive multi-arm polymer (120) and the amino groups of the therapeutic-agent-releasing polyamino compound (118) is suppressed. Then, when crosslinking is desired, a pH of the mixture of the therapeutic-agent-releasing polyamino compound (118) and the reactive multi-arm polymer (120) is changed from an acidic pH to a basic pH, leading to crosslinking between same, thereby forming the crosslinked product (130).

In particular embodiments, the system comprises (a) a first composition that comprises a therapeutic-agent-releasing polyamino compound as described hereinabove, (b) a second composition that comprises a reactive multi-arm polymer as described hereinabove, and (c) a third composition, specifically, an accelerant composition, that contains an accelerant that is configured to accelerate crosslinking reaction between the therapeutic-agent-releasing polyamino compound and the reactive multi-arm polymer.

The first composition may be a first fluid composition comprising the therapeutic-agent-releasing polyamino compound that is buffered to an acidic pH or a first dry composition that comprises the therapeutic-agent-releasing polyamino compound and acidic buffering composition, to which a suitable fluid such as water for injection, saline, etc. can be added to form a first fluid composition comprising the therapeutic-agent-releasing polyamino compound that is buffered to an acidic pH. In some embodiments, for example, the acidic buffering composition may comprise monobasic sodium phosphate, among other possibilities. The first fluid composition comprising the therapeutic-agent-releasing polyamino compound may have a pH ranging, for example, from about 3 to about 5, typically ranging from about 3.5 to about 4.5, and more typically ranging from about 3.8 to about 4.2. In addition to the therapeutic-agent-releasing polyamino compound, the first composition may further comprise additional agents, such as imaging agents and/or colorants.

The second composition may be a second fluid composition comprising the reactive multi-arm polymer or a second dry composition that comprises the reactive multi-arm polymer from which a fluid composition is formed, for example, by the addition of a suitable fluid such as water for injection, saline, or the first fluid composition comprising the therapeutic-agent-releasing polyamino compound that is buffered to an acidic pH. In addition to the reactive multi-arm polymer, the second composition may further comprise additional agents, such as imaging agents and/or colorants.

In a particular embodiment, the first composition is a first fluid composition comprising the therapeutic-agent-releasing polyamino compound that is buffered to an acidic pH and the second composition comprises a dry composition that comprises the reactive multi-arm polymer. The first composition may then be mixed with the second composition to provide a prepared fluid composition that is buffered to an acidic pH and comprises the therapeutic-agent-releasing polyamino compound and the reactive multi-arm polymer. In a particular example, a syringe may be provided that contains a first fluid composition comprising the therapeutic-agent-releasing polyamino compound that is buffered to an acidic pH, and a vial may be provided that comprises a dry composition (e.g., a powder) that comprises the reactive multi-arm polymer. The syringe may then be used to inject the first fluid composition into the vial containing the reactive multi-arm polymer to form a prepared fluid composition that contains the therapeutic-agent-releasing polyamino compound and the reactive multi-arm polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11, typically ranging from about 9.5 to about 10.5, and more typically ranging from about 9.8 to about 10.2. In addition to the above, the fluid accelerant composition may further comprise additional agents, such as imaging agents and/or colorants.

Examples of imaging agents include radiocontrast agents, imageable radioisotopes, fluorescent dyes, magnetic resonance imaging (MRI) contrast agents, ultrasound contrast agents and near-infrared (NIR) imaging contrast agents. Particular examples of radiocontrast agents include metallic particles such as particles of tantalum, tungsten, rhenium, niobium, gold, molybdenum, and their alloys, which metallic particles may be spherical or non-spherical. Particular examples of radiocontrast agents further include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®). Further particular examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, and (e) imageable radioisotopes including ⁹⁹mTc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr and ¹⁷⁷Lu, among others.

Examples of additional agents also include colorants such as brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

A prepared fluid composition that is buffered to an acidic pH and comprises the therapeutic-agent-releasing polyamino compound and the reactive multi-arm polymer as described above, and a fluid accelerant composition that is buffered to basic pH as described above, may be combined form crosslinked hydrogels, either in vivo or ex vivo.

In various embodiments, a system is provided that include one or more delivery devices for delivering first and second compositions to a subject.

In some embodiments, the system may include a delivery device that comprises a first reservoir that contains a first composition that comprises a therapeutic-agent-releasing polyamino compound as described above and a second reservoir that contains a second composition that comprises a reactive multi-arm polymer that comprises a plurality of reactive end groups that are reactive with the amino groups of the therapeutic-agent-releasing polyamino compound as described above.

In some embodiments, the system may include a delivery device that comprises a first reservoir that contains a first composition that comprises the therapeutic-agent-releasing polyamino compound and the reactive multi-arm polymer and is buffered to an acidic pH, such as the prepared fluid composition previously described, and a second reservoir that contains second composition, such as the fluid accelerant composition previously described. In either case, during operation, the first composition and second composition are dispensed from the first and second reservoirs and combined, whereupon the therapeutic-agent-releasing polyamino compound and the reactive multi-arm polymer and crosslink with one another to form a hydrogel.

Regardless of the first and second compositions selected, in particular embodiments, the system may include a delivery device that comprises a double-barrel syringe, which includes first barrel having a first barrel outlet, which first barrel contains the first composition, a first plunger that is movable in the first barrel, a second barrel having a second barrel outlet, which second barrel contains the second composition, and a second plunger that is movable in the second barrel.

In some embodiments, the device may further comprise a mixing section having a first mixing section inlet in fluid communication with the first barrel outlet, a second mixing section inlet in fluid communication with the second barrel outlet, and a mixing section outlet. In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-releasing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

In other embodiments, the therapeutic-agent-releasing hydrogels of the present disclosure comprise a therapeutic-agent-releasing polysaccharide that comprises polysaccharide moiety and a plurality of therapeutic agent precursors that are covalently linked along a backbone of the polysaccharide moiety. In some of these embodiments, the therapeutic agent precursors are covalently linked to the polysaccharide moiety through ester groups. Upon hydrolysis of the ester bond, the therapeutic agent is released from the polysaccharide moiety.

In various embodiments, the therapeutic agent precursors are precursors of hydroxyl-substituted therapeutic agents. In these embodiments, upon hydrolysis of the ester bond, the therapeutic-agent-releasing polysaccharide releases the hydroxyl-substituted therapeutic agent. As seen below, in various embodiments, the therapeutic agent precursor is a residue of a hydroxyl-substituted therapeutic agent, which may be formed, for example, by an ester-forming reaction between a hydroxyl-substituted therapeutic agent and a carboxylic-acid-containing polysaccharide.

In various embodiments, the polysaccharide moiety is derived from a carboxylic-acid-containing polysaccharide. Carboxylic-acid-containing polysaccharides include those that contain one or more uronic acid species, such as galacturonic acid, glucuronic acid and/or iduronic acid. Particular examples of carboxylic-acid-containing polysaccharides include alginic acid, hyaluronic acid, pectin, agaropectin, carrageenan, gellan gum, gum arabic, guar gum, xanthan gum, and carboxymethyl cellulose moieties. In embodiments where the carboxylic-acid-containing polysaccharide is hyaluronic acid, the carboxylic-acid-containing polysaccharide may be non-animal stabilized hyaluronic acid. In some embodiments, the carboxylic-acid-containing polysaccharides may have a number average molecular weight ranging from 1 kDa to 8000 kDa, for example ranging anywhere from 1 kDa to 2.5 kDa to 5 kDa to 10 kDa to 25 kDa to 50 kDa to 100 kDa to 250 kDa to 500 kDa to 1000 kDa to 2000 kDa to 8000 kDa (in other words, ranging between any two of the preceding numerical values).

The therapeutic-agent-releasing polysaccharide compositions of the present disclosure may be formed by an ester coupling reaction between a carboxylic-acid containing polysaccharide, such as one of those described above, among others, and a hydroxyl-substituted therapeutic agent, such as one of those described above, among others. Such a coupling reaction may be performed using a suitable coupling reagent, for instance, a carbodiimide coupling reagent. In a particular example, hyaluronic acid or alginic acid may be employed as the carboxylic-acid containing polysaccharide and doxorubicin may be employed as the hydroxyl-substituted therapeutic agent. 1-ethyl-3-(3-dimethylaminopropyl)carbodiimide (EDC) may be used as a coupling reagent.

The therapeutic-agent-releasing polysaccharide may be in the form of a hydrogel when hydrated.

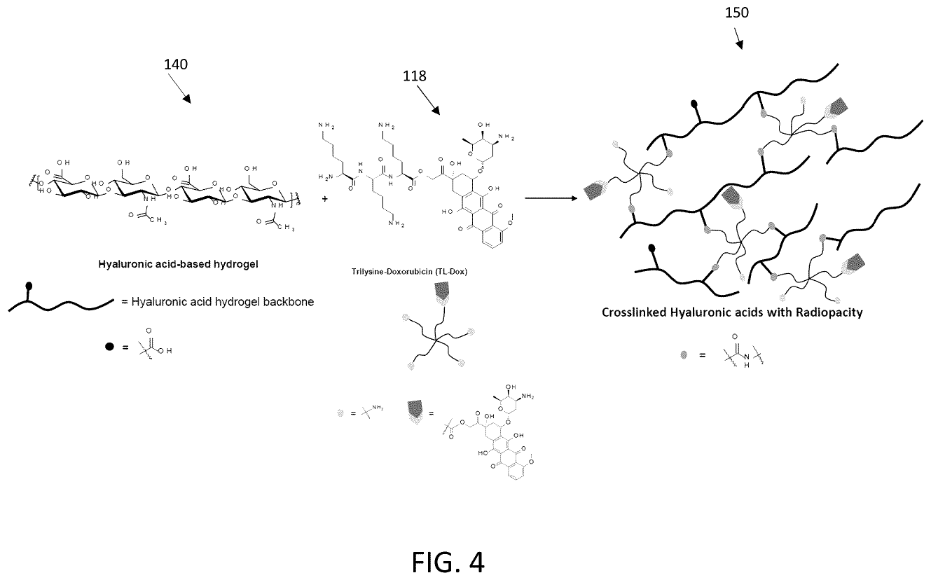

The therapeutic-agent-releasing polysaccharide compositions of the present disclosure may also be formed by an amide coupling reaction between a carboxylic-acid containing polysaccharide, such as one of those described above, among others, and a therapeutic-agent-releasing polyamino compound, such as one of those described above, among others. Such a coupling reaction may be performed using a suitable coupling reagent, for instance, a carbodiimide coupling reagent. In a particular example shown in FIG. 4, hyaluronic acid (140) may be employed as the carboxylic-acid containing polysaccharide and trilysine-doxorubicin conjugate (TL-Dox) (118) may be employed as the therapeutic-agent-releasing polyamino compound, to form a therapeutic-agent-releasing crosslinked polysaccharide product (150), which may be in the form of a hydrogel when hydrated.

In addition to a therapeutic-agent-releasing polysaccharide as described above, therapeutic-agent-releasing hydrogel compositions in accordance with the present disclosure may contain additional agents, such as imaging agents and/or colorants as described above.

In various embodiments, kits are provided that include one or more delivery devices for delivering the therapeutic-agent-releasing polysaccharide to a subject. Such systems may include one or more of the following: a syringe barrel, which may or may not contain a therapeutic-agent-releasing polysaccharide as described herein; a vial, which may or may not contain a therapeutic-agent-releasing polysaccharide as described here; a needle; a flexible tube (e.g., adapted to fluidly connect the needle to the syringe), an injectable liquid such as water for injection, normal saline or phosphate buffered saline. Whether supplied in a syringe, vial, or other reservoir, the therapeutic- agent-releasing polysaccharide may be provided in dry form (e.g., powder form) or in a form that is ready for injection, such as an injectable hydrogel form.

The therapeutic-agent-releasing polysaccharide hydrogels described herein can be used for a number of purposes.

For example, therapeutic-agent-releasing polysaccharide hydrogels can be injected to provide spacing between tissues, therapeutic-agent-releasing polysaccharide hydrogels can be injected (e.g., in the form of blebs) to provide fiducial markers, therapeutic-agent-releasing polysaccharide hydrogels can be injected for tissue augmentation or regeneration, therapeutic-agent-releasing polysaccharide hydrogels can be injected as a filler or replacement for soft tissue, therapeutic-agent-releasing polysaccharide hydrogels can be injected to provide mechanical support for compromised tissue, therapeutic-agent-releasing polysaccharide hydrogels be injected as a scaffold, and/or therapeutic-agent-releasing polysaccharide hydrogels can be injected as a carrier of therapeutic agents in the treatment of diseases and cancers and the repair and regeneration of tissue, among other uses.

After administration, the compositions of the present disclosure can be imaged using a suitable imaging technique.

As seen from the above, the therapeutic-agent-releasing polysaccharide hydrogels of the present disclosure may be used in a variety of medical procedures, including the following, among others: a procedure to implant a fiducial marker comprising a therapeutic-agent-releasing polysaccharide hydrogel, a procedure to implant a tissue regeneration scaffold comprising a therapeutic-agent-releasing polysaccharide hydrogel, a procedure to implant a tissue support comprising a therapeutic-agent-releasing polysaccharide hydrogel, a procedure to implant a tissue bulking agent comprising a therapeutic-agent-releasing polysaccharide hydrogel, a procedure to implant a therapeutic-agent-containing depot comprising a therapeutic-agent-releasing polysaccharide hydrogel, a tissue augmentation procedure comprising implanting a therapeutic-agent-releasing polysaccharide hydrogel, a procedure to introduce a therapeutic-agent-releasing polysaccharide hydrogel between a first tissue and a second tissue to space the first tissue from the second tissue.

The polysaccharide hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

What is claimed is:

1. A therapeutic-agent-releasing polyamino compound comprising polyamino moiety that is covalently linked to a residue of a hydroxyl-substituted therapeutic agent by an ester bond.

2. The therapeutic-agent-releasing polyamino compound of claim 1, wherein hydrolysis of the ester bond results in formation of the hydroxyl-substituted therapeutic agent and a carboxyl-substituted polyamino compound.

3. The therapeutic-agent-releasing polyamino compound of claim 1, wherein the hydroxyl-substituted therapeutic agent comprises one or more primary hydroxyalkyl groups selected from hydroxymethyl, 2-hydroxyethyl, 3-hydroxypropyl, 4-hydroxybutyl, 5-hydroxypentyl, 6-hydroxyhexyl, 7-hydroxyheptyl and/or 8-hydroxyoctyl groups.

4. The therapeutic-agent-releasing polyamino compound of claim 1, wherein the polyamino moiety comprises a plurality of —(CH₂)ₓ—NH₂ groups where x is 0, 1, 2 3, 4, 5, 6, 7 or 8.

5. The therapeutic-agent-releasing polyamino compound of claim 1, wherein the polyamino moiety comprises a residue of a carboxyl-substituted polyamino compound.

6. The therapeutic-agent-releasing polyamino compound of claim 1, wherein the polyamino moiety comprises two or more amino acid residues selected from residues of lysine, ornithine, and combinations thereof.

7. A system for forming a hydrogel that comprises a therapeutic-agent-releasing polyamino compound of claim 1 and a reactive multi-arm polymer that comprises a plurality of hydrophilic polymer arms having reactive end groups that are reactive with amino groups of the therapeutic-agent-releasing polyamino compound.

8. The system of claim 7, wherein the hydrophilic polymer arms comprise one or more hydrophilic monomers selected from ethylene oxide, N-vinyl pyrrolidone, oxazolines, hydroxyethyl acrylate, hydroxyethyl methacrylate, PEG methyl ether acrylate or PEG methyl ether methacrylate, or PNIPAAM.

9. The system of claim 7, wherein the reactive end groups are linked to the hydrophilic polymer arms by a hydrolysable ester and/or wherein the reactive end groups are electrophilic groups.

10. The system of claim 9, wherein the electrophilic groups are selected from imidazole esters, imidazole carboxylates, benzotriazole esters, or imide esters.

11. The system of claim 7, wherein the system comprises a first composition that comprises the therapeutic-agent-releasing polyamino compound, a second composition that comprises the reactive multi-arm polymer, and an optional accelerant composition.

12. The system of claim 7, further comprising a delivery device.

13. A medical hydrogel formed by crosslinking the therapeutic-agent-releasing polyamino compound in accordance with claim 1 and a reactive multi-arm polymer that comprises a plurality of hydrophilic polymer arms having reactive end groups that are reactive with amino groups of the therapeutic-agent-releasing polyamino compound.

14. A method of treatment comprising administering to a subject a mixture that comprises a therapeutic-agent-releasing polyamino compound in accordance with claim 1 and a reactive multi-arm polymer that comprises a plurality of hydrophilic polymer arms having reactive end groups that are reactive with amino groups of the therapeutic-agent-releasing polyamino compound under conditions such that the therapeutic-agent-releasing polyamino compound and the reactive multi-arm polymer cross-link after administration to the subject.

15. A method of making a therapeutic-agent-releasing polyamino compound comprising (a) forming a protected carboxyl-substituted polyamino compound by protecting amino groups of the carboxyl-substituted polyamino compound, (b) forming an ester linkage between the carboxyl group of the protected carboxyl-substituted polyamino compound and a primary hydroxyl group of a hydroxyl-containing therapeutic agent and (c) deprotecting amino groups of the product of step (b).

16. A therapeutic-agent-releasing polysaccharide that comprises a plurality of hydroxyl-substituted therapeutic agent residues that are covalently linked to a carboxylic-acid-containing polysaccharide along a backbone of the carboxylic-acid-containing polysaccharide.

17. The therapeutic-agent-releasing polysaccharide of claim 16, wherein the therapeutic-agent-releasing moieties are covalently linked to the carboxylic-acid-containing polysaccharide through ester bonds.

18. The therapeutic-agent-releasing polysaccharide of claim 16, wherein the carboxylic-acid-containing polysaccharide comprises one or more uronic acid species selected from galacturonic acid, glucuronic acid, and iduronic acid.

19. The therapeutic-agent-releasing polysaccharide of claim 16, wherein the carboxylic-acid-containing polysaccharide is selected from hyaluronic acid, alginic acid, pectin, agaropectin, carrageenan, gellan gum, gum arabic, guar gum, xanthan gum, and carboxymethyl cellulose.

20. A method of treatment comprising administering to a subject a therapeutic-agent-releasing polysaccharide in accordance with claim 16.
